(19) United States
(12) Patent Application Publication
(10) Pub. No.: US 2008/0114440 A1
(43) Pub. Date: May 15, 2008

(54) Methods and devices for deploying an implant in curved anatomy

(75) Inventors: Edwin J. Hlavka, Minneapolis, MN (US); Alex Alden Peterson, Maple Grove, MN (US)

Correspondence Address: O''Melveny & Myers LLP;IP&T Calendar Department LA-1118, 400 South Hope Street, Los Angeles, CA, 90071-2899, US

(73) Assignee: Sage Medical Technologies, Inc

(21) Appl. No.: 11/605,594

(22) Filed: Nov. 27, 2006

Related U.S. Application Data
(60) Provisional application No. 60/858,621, filed on Nov. 13, 2006.

(51) Int. Cl. A61F 2/84

(52) U.S. Cl. 623/1.12; 623/1.11

(57) ABSTRACT

Methods and devices for deploying an endoluminal implant in a curved vessel. The devices include steerable catheters having a control member that extends to the distal end and operates to bend the catheter. In other cases, the stent includes a longitudinal adjustment member attached near the leading edge of the stent and operates to shorten or lengthen the stent and thereby induce a curvature. The stent is carried in a distal region of the catheter that is advanced into a curved region of a vessel. The catheter is bent and/or the stent is curved using the longitudinal adjustment member, and the stent is deployed to achieve uniform wall contact with the endoluminal surface of the vessel at the lesser curvature.

### RELATED APPLICATION

This application claims the benefit of U.S. Provisional Patent Application Ser. No. 60/858,621, filed Nov. 13, 2006. The entire content of this application is incorporated herein by reference in its entirety.

### FIELD OF THE INVENTION

The present invention relates generally to endoluminal implants and deployment thereof in curved anatomy by steering the deployment catheter and/or the implant to conform to the vessel curvature and thereby achieve uniform wall contact.

### BACKGROUND

Aortic dissection most commonly occurs in patients between the ages of 40 to 60 years old and is two or three times more frequent in men than women within this age group. Hypertension, a coexisting condition in 70% of the patients, is almost invariably the most important factor causing or initiating aortic dissection. Other risk factors that predispose a patient to develop aortic dissection include aortic dilation, aortic aneurysm, congenital valve abnormality, coarctation of aorta, and Marfan syndrome. These patients often present with sudden, severe, and tearing pain that may be localized in the front or back of the chest. Other symptoms include syncope, dyspnea, and weakness. These presentations are the consequence of intimal tear in the aorta, dissecting hematoma, occlusion of involved arteries, and compression of adjacent tissues. For example, patients may have neurological symptoms, such as hemiplegia, due to carotid artery obstruction, or paraplegia, due to spinal cord ischemia. Patients may also present with bowel ischemia or cardiac ischemia due to occlusion of major arteries by the dissecting aorta.

Aortic dissection can be classified by the Stanford method into type A and type B depending on the location and the extent of the dissection. Type A dissection, or proximal dissection, involves the ascending aorta. Type B dissection, or distal dissection, usually begins just downstream of the left subclavian artery, extending downward into the descending and abdominal aorta. If left untreated, the risk of death from aortic dissection can reach 35% within 15 minutes after onset of symptoms and 75% by one week.

Once diagnosed, aortic dissection is treated with immediate medical management aimed at reducing cardiac contractility and systemic arterial pressure, thereby reducing shear stress on the aorta. Beta-adrenergic blockers, unless contraindicated, are usually used to treat acute dissection. Surgical correction, including reconstruction of the aortic wall, is usually the preferred treatment for ascending aortic dissection (type A). Medical therapy is the preferred treatment for stable and uncomplicated distal aortic dissection (type B), unless there is clinical evidence of propagation, obstruction of major arterial branches, or impending aortic rupture in which case surgical correction is preferred. In-hospital mortality for medically treated patients with type B dissection is between 15 to 20 percent. Morbidity and mortality for surgical correction is not significantly better than medically treated patients. Currently, there is no good treatment for type B aortic dissection. A need for devices and methods therefore exists to treat patients suffering from Type B dissection.

### SUMMARY OF THE INVENTION

The present invention relates to devices and methods for deploying an endoluminal implant, e.g., a stent, in curved anatomy, e.g., a curved vessel. More particularly, the devices are steerable catheters and/or steerable stents. The catheter is advanced into a curved region of the vessel, the vessel having a centerline. The catheter is curved to (1) substantially align a longitudinal axis of the catheter with the centerline of the vessel at the region where the implant is to be deployed in the vessel, (2) substantially align the longitudinal axis of the catheter parallel with a tangent to the centerline of the vessel, (3) substantially align the longitudinal axis of the catheter parallel with a tangent to the wall of the vessel, or (4) achieve an orientation relative to the vessel curvature desired by the physician. The implant is then deployed to achieve substantially uniform wall contact with the endoluminal surface of the vessel.

In certain cases, the curved vessel is the aorta, more particularly the aortic arch. The catheter may be advanced upstream of the innominate artery, downstream of the innominate artery, upstream of the left subclavian artery, or downstream of the left subclavian artery. The catheter and the implant can be placed to overlap the entry point of an aortic dissection. In other cases, the implant is placed to cover an aortic atheroma, e.g., a mobile aortic atheroma, and hold the atheroma in place between the implant and the endoluminal surface of the aorta.

The steerable catheter may include a control member attached to a point on the catheter near the distal end and extending proximately from the point of the attachment. The catheter may be deflected by operating the control member, e.g., by withdrawing or advancing the control member. The control member may be attached to a point on the circumference of the catheter. Numerous other designs for steerable catheters are well known to those skilled in the art and will be understood to be suitable for use in the present invention. The use of a control member is therefore merely illustrative of one design that can be used in the present invention.

The stent may be carried near the distal end of the catheter. In certain cases, the stent is a self-expanding stent made from a superelastic material, e.g., nitinol or laser-etched nitinol. In other cases, the stent will include a textile, e.g., a porous textile, covering all or a portion of the stent. Textiles may be used to promote cellular ingrowth and healing of the vessel. The stent may be released and deployed by withdrawing a catheter sheath or the catheter itself to release the stent. A pusher or stylet may be used to hold the stent in place so that the stent is not withdrawn as the catheter is pulled back.

The present invention also contemplates endoluminal implants having a longitudinal adjustment member. The longitudinal adjustment member is attached on the implant near the leading edge and extends proximately from the point of attachment. The endoluminal implant is carried in a distal region of the catheter, and the catheter is advanced into a curved region of a vessel. The implant is deployed in the vessel. The longitudinal adjustment member is moved to adjust the orientation of a plane defined by the leading edge of the endoluminal implant so that the endoluminal implant achieves uniform wall contact with the endoluminal surface of the vessel where the endoluminal implant engages the lesser curvature of the vessel. The longitudinal adjustment member can be moved to adjust orientation before, during, or after deployment of the endoluminal implant.

The longitudinal adjustment member can be fixedly or releasably attached to the endoluminal implant at the leading edge. In certain cases, the longitudinal adjustment member extends proximally to a point of attachment near the trailing edge of the endoluminal implant. In other cases, the longitudinal adjustment member has a distal segment, a proximal segment, and an adjustable mechanism, e.g., a cinching mechanism disposed between the proximal and distal segments. The adjustable mechanism operates to shorten or lengthen the longitudinal adjustment member to adjust the radius of curvature of the endoluminal implant. In this way adjustment causes the endoluminal implant to (1) substantially align a longitudinal axis of the endoluminal implant with the centerline of the vessel, (2) substantially align the longitudinal axis of the endoluminal implant parallel with a tangent to the centerline of the vessel, (3) substantially align the longitudinal axis of the endoluminal implant parallel with a tangent to the wall of the vessel, or (4) achieve an orientation relative to the vessel curvature desired by the physician.

### BRIEF DESCRIPTION OF THE DRAWINGS

FIG. 1A depicts a longitudinal cross-section of a pre-curved catheter advanced into the aortic arch.

FIG. 1B depicts a longitudinal cross-section of a catheter deploying a stent in the descending aorta.

FIG. 2 depicts a steerable catheter for use in stent deployment.

FIG. 3A depicts a longitudinal cross-section of a steerable catheter curved to an orientation parallel to the centerline in the aortic arch.

FIG. 3B depicts a longitudinal cross-section of the steerable catheter deploying a stent in the descending aorta.

FIG. 4A depicts a longitudinal cross-section of a controllable stent placed in the descending aorta.

FIG. 4B depicts a longitudinal cross-section of the controllable stent of FIG. 4A adjusted to an orientation that conforms to the centerline in the aortic arch.

FIG. 5A depicts a longitudinal cross-section of a stent having first and second longitudinal adjustment numbers placed in the descending aorta.

FIG. 5B depicts a longitudinal cross-section of the stent of FIG. 5A adjusted to an orientation that conforms to the centerline in the aortic arch.

FIG. 6 depicts a longitudinal cross-section of an alternative controllable stent with releasable control mechanism placed in the descending aorta.

### DETAILED DESCRIPTION

The devices and methods described herein facilitate stent deployment in a curved or tortuous vascular anatomy to ensure uniform wall contact between the stent and the endoluminal surface of the vessel. This result may be achieved by actively steering the stent-delivery catheter, the stent itself, both the catheter and the stent, and by other techniques described herein. The catheter and/or the stent may be adjusted in certain cases to substantially align with the longitudinal axis of the catheter and/or stent with the centerline of the vessel at the region where the implant lies within the vessel. In other cases, the catheter and/or the stent may be adjusted to substantially align the longitudinal axis parallel with a tangent to the wall of the vessel at the region where the implant lies within the vessel. In still other cases, the catheter and/or the stent may be adjusted to substantially align the longitudinal axis parallel with a tangent to the centerline of the vessel at the region where the implant lies within the vessel.

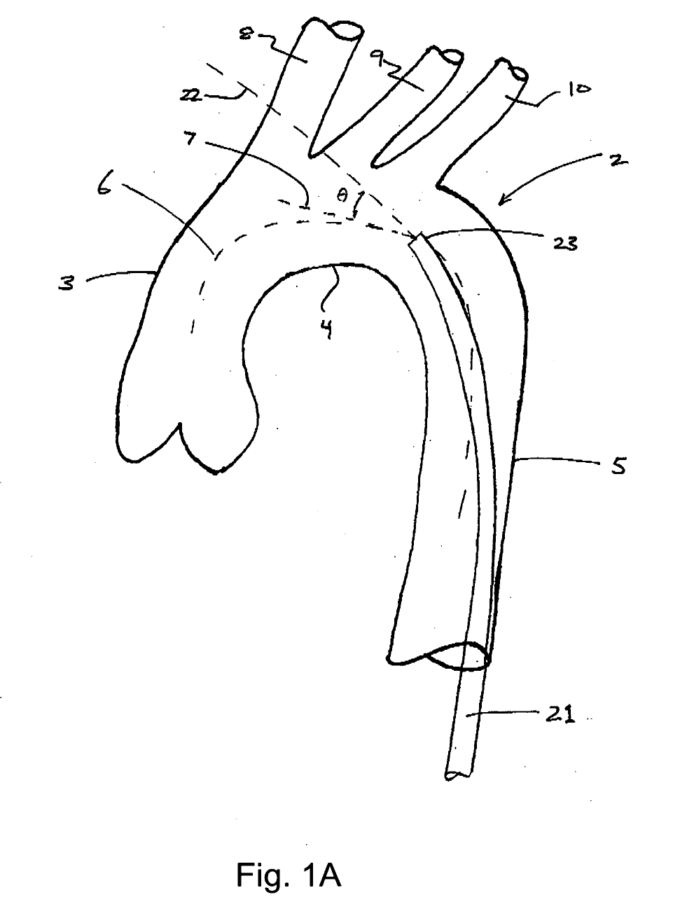

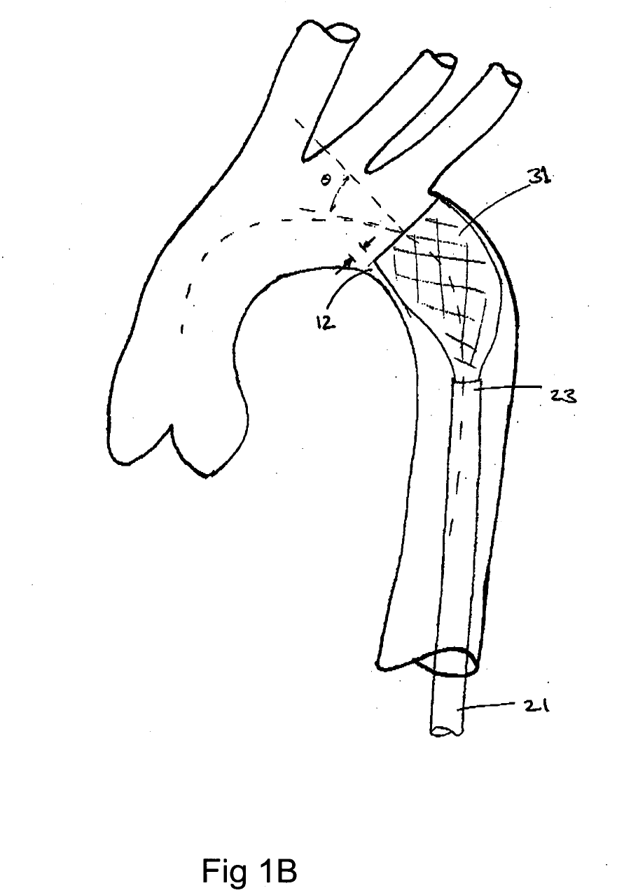

FIG. 1A depicts a frontal view of an aorta 2, which is described as including ascending aorta 3, aortic arch 4, and descending aorta 5. Innominate artery 8, common carotid artery 9, and left subclavian artery 10 branch from aortic arch 4 and supply blood to the brain and other organs. The lumen of aortic arch 4 defines a curve having centerline 6. Catheter 21 is shown advanced retrograde through the descending aorta so that distal end 23 lies within the aortic arch. Distal end 23 of catheter 21 lies within aortic 2 at a point on centerline 6 having tangent line 7. In cases where catheter 21 is straight or pre-curved but does not match the vessel curvature at the point of placement, longitudinal axis 22 of catheter 21 at distal end 23 is displaced by angle θ relative to tangent line 7. If, as shown in FIG. 1B, a stent 31 is then deployed in this curved vessel at displacement angle θ, gap 12 will occur between the leading edge of stent 31 and the endoluminal surface of aorta 2 at the lesser curvature. Blood flow around the lesser curvature of aortic arch 4 impacts the leading edge of stent 31, creating turbulence in increasing the gap 12.

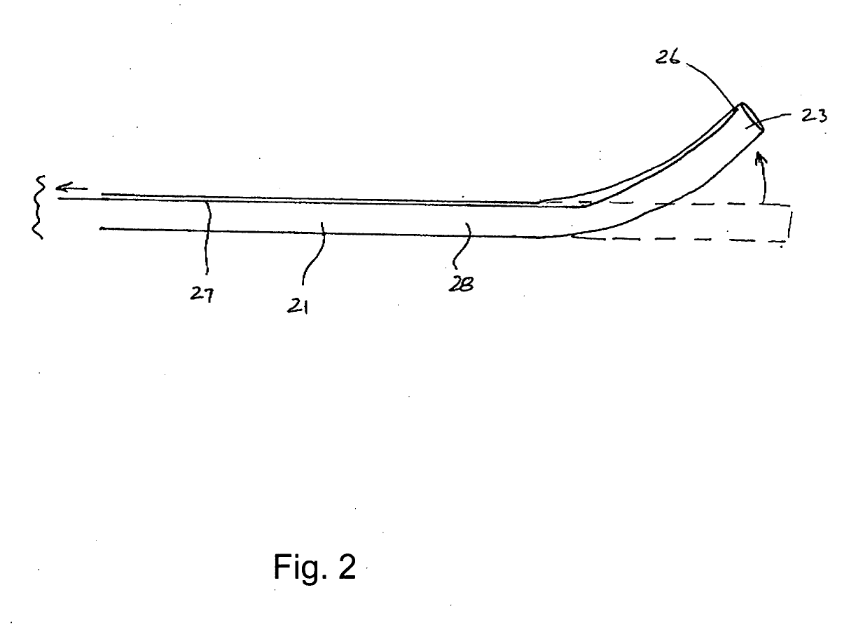

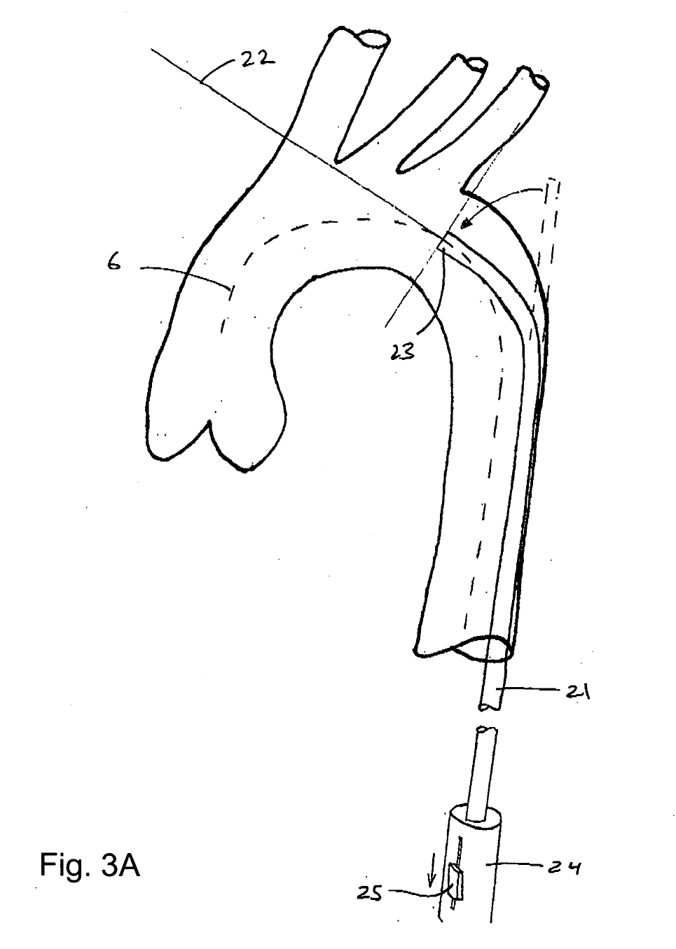

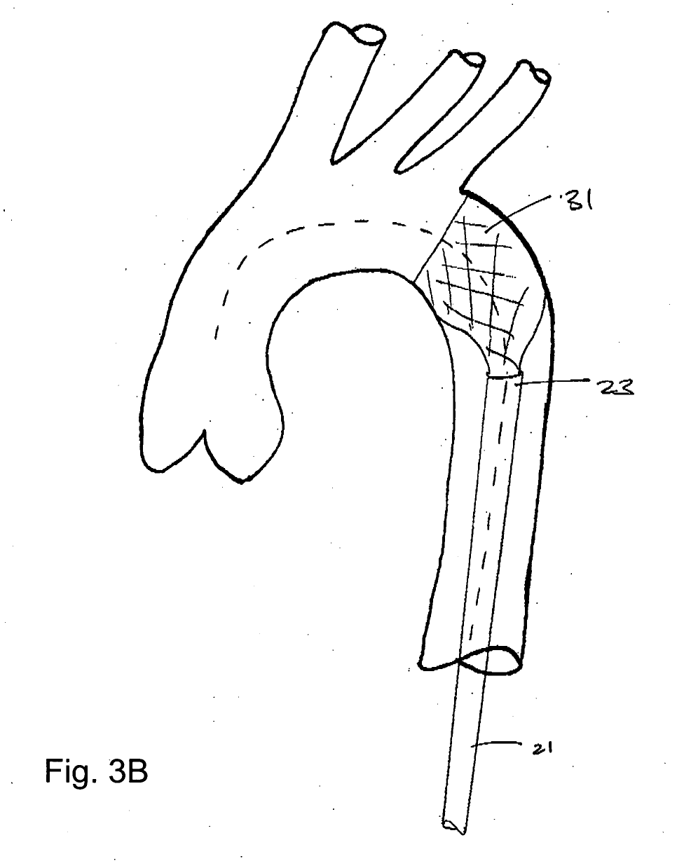

A steerable catheter for use herein is depicted in FIG. 2. Catheter 21 has proximal end, distal end 23, and lumen 28 adapted to carry a stent or any other endoluminal implant. Catheter 21 may, in certain cases, include control member 27, e.g., a control wire, which is bonded to catheter 21 at attachment point 26 near distal end 23. Control member 27 may extend proximally from attachment point 26 to control handle 24, operable at the proximal end of catheter 21 as shown in FIG. 3A. Withdrawing control member 27 causes the distal end of catheter 21 to curve in use.

In use, catheter 21 advances into a curved vessel, e.g., descending aorta 5 as depicted in FIG. 3. Catheter 21 is positioned in a region of interest, e.g., at the entry point of an aortic dissection or a region having a lesion or atheroma, e.g., an aortic atheroma or a mobile aortic atheroma. The procedure may be conducted using standard fluoroscopic visualization techniques to align catheter 21 with anatomical landmarks visible by angiography. One or more fluoroscopic markers may be included on catheter 21, on the distal region or distal end 23 of catheter 21, and/or on stent 31 for purposes of alignment. The takeoff of left subclavian artery or the entry point of a dissection are among anatomical landmarks useful for alignment.

Control mechanism 25 on control handle 24 may be operated to deflect distal end 23 of catheter 21. The distal end of catheter 21 is deflected relative to the centerline of the vessel to (1) substantially align the longitudinal axis of catheter 21 with the centerline of the vessel, (2) substantially align the longitudinal axis of catheter 21 parallel with a tangent to the centerline of the vessel, (3) substantially align the longitudinal axis of catheter 21 parallel with a tangent to the wall of the vessel, or (4) achieve an orientation relative to the vessel curvature desired by the physician. As depicted in FIG. 3B, stent 31 is then deployed by withdrawing catheter 21 or a capture sheath to release the endoluminal implant. Because stent 31 is aligned with the vessel curvature when it expands, stent 31 achieves uniform wall contact when deployed. This technique eliminates or reduces any gap between the leading edge of stent 31 and the endoluminal surface of the lesser curvature of the curved vessel.

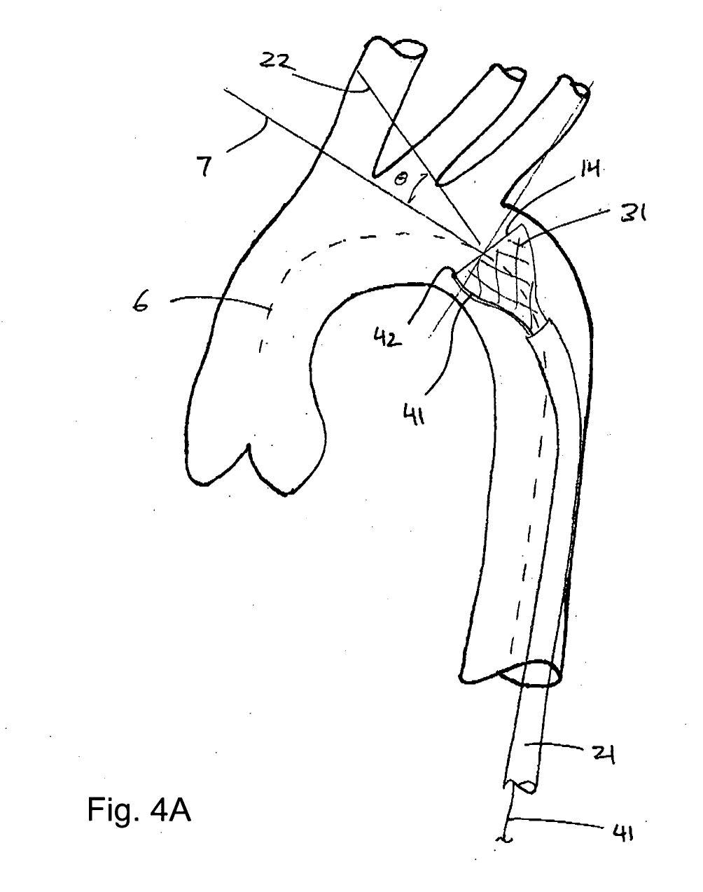

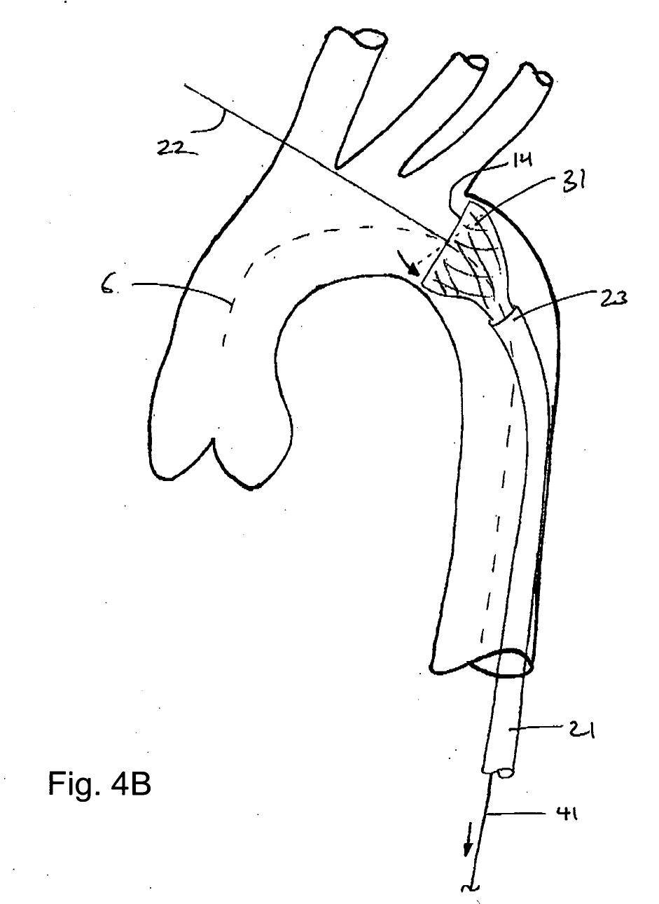

In addition to, or instead of steering the catheter, the positioning of the stent itself can be actively controlled before, during, and/or after deployment as depicted in FIG. 4A. Stent 31 may include longitudinal adjustment member 41 that extends proximately from attachment point 42 at the leading edge. By withdrawing adjustment member 41 (when attachment point 42 is near the lesser curvature) or by extending adjustment member 41 (when attachment point 42 is near the greater curvature), a plane 14 defined by the leading edge of the stent is adjusted in orientation relative to tangent 7 to vessel centerline 6 as depicted in FIG. 4B. Orientation of the leading edge of the stent is adjusted before, during, or after deployment. The adjustment causes the stent to (1) substantially align a longitudinal axis of the stent with the centerline of the vessel, (2) substantially align the longitudinal axis of the stent parallel with a tangent to the centerline of the vessel, (3) substantially align the longitudinal axis of the stent parallel with a tangent to the wall of the vessel, or (4) achieve an orientation relative to the vessel curvature desired by the physician.

As depicted in FIG. 4B, stent 31 is then deployed by withdrawing catheter 21 to release the endoluminal implant. Because stent 31 is aligned with the vessel curvature, stent 31 achieves uniform wall contact when deployed. This device and method eliminates or reduces any gap between the leading edge of stent 31 and the endoluminal surface of the lesser curvature of the curved vessel.

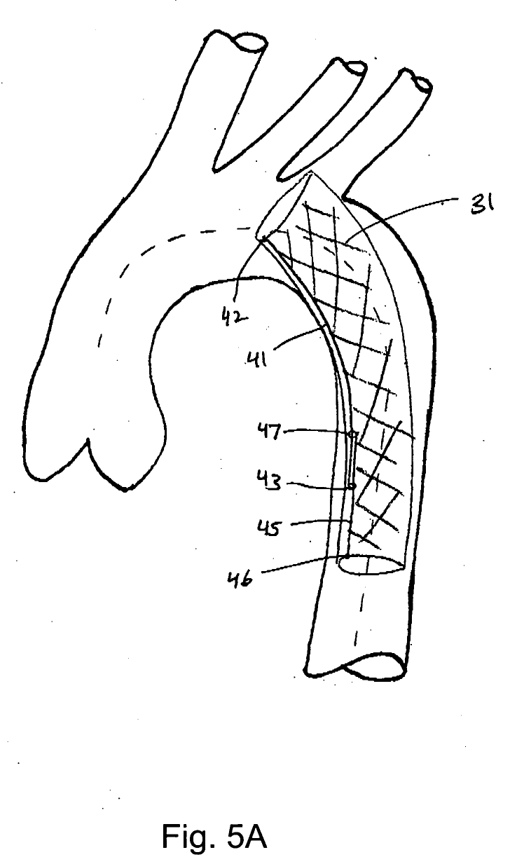

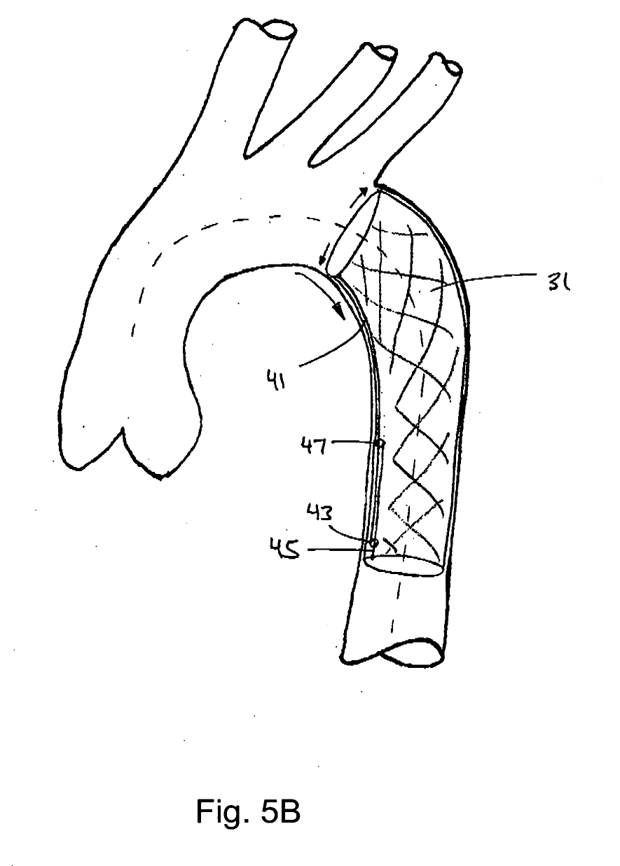

Adjustment member 41 can be fixedly attached near the leading edge of stent 31 or, alternatively, releasably attached near the leading edge. When releasably attached, the adjustment member may be removed after the stent is deployed and desired placement is established. Adjustment member 41 may extend proximally within the catheter to near the proximal end of the catheter or it may be attached to a position at the trailing edge of stent 31. As depicted in FIG. 5A, the adjustment member may comprise first adjustment member 41 and second adjustment member 45 where the first and second adjustment members are slideably connected at an intermediate region on stent 31. Adjustment member 41 may be fixedly attached at the leading edge and extend proximally to cinching mechanism 43, e.g., a loop. Adjustment member 45 may be fixedly attached at the trailing edge and extend distally, interacting with cinching mechanism 43, and optionally extending to cinching mechanism 47. In use, the curvature of stent 31 is adjusted by reducing or lengthening the adjustment member as depicted in FIG. 5B.

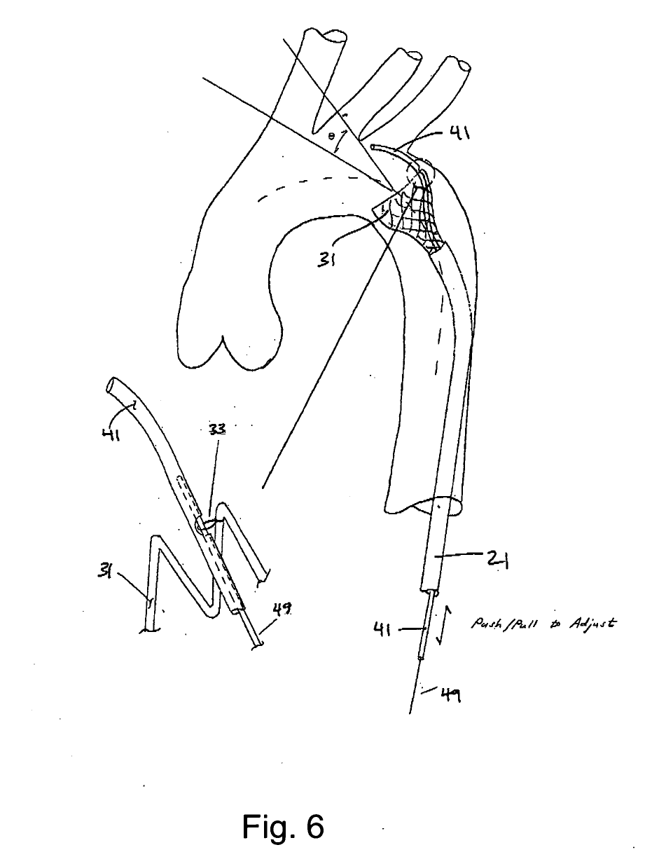

Adjustment member 41 is depicted in FIG. 6 in a further alternative as releasably or fixedly attached to the leading edge of stent 31 and substantially aligned on the greater curvature. The orientation of the plane defined by the leading edge of the stent is adjusted by moving the adjustment member proximally or distally. Adjustment member 41 carries retention element 49, which may be slideably disposed in a lumen of adjustment member 41. Retention element 49 engages loop 33 on the leading edge of stent 31. After the orientation of the plane of the leading edge is adjusted to (1) substantially align a longitudinal axis of the stent with the centerline of the vessel, (2) substantially align the longitudinal axis of the stent parallel with a tangent to the centerline of the vessel, (3) substantially align the longitudinal axis of the stent parallel with a tangent to the wall of the vessel, or (4) achieve an orientation relative to the vessel curvature desired by the physician, retention element 49 may be withdrawn proximally to release loop 33. Adjustment member 41 is thereby disengaged from stent 31. Adjustment member 41 is then removed from the patient.

The working length of catheter 21 will generally be between 30 and 100 centimeters, preferably approximately between 50 and 80 centimeters. The outer diameter of the catheter 21 shaft will generally be between 5 French and 25 French, preferably approximately between 10 French and 16 French. Stent 31 may vary in length but is generally approximately 5 cm to 30 cm, preferably approximately 10 cm to 20 cm. The foregoing ranges are set forth solely for the purpose of illustrating typical device dimensions. The actual dimensions of a device constructed according to the principles of the present invention may obviously vary outside of the listed ranges without departing from those basic principles.

Although the foregoing invention has, for purposes of clarity and understanding, been described in some detailed by way of illustration and example, it will be obvious that certain changes and modifications may be practiced that will still fall within the scope of the attended claims. Moreover, although certain features have been depicted in one figure or with reference to one embodiment, it is understood that the features depicted in any one implementation can be used in combination with features in any other implementation or figure.

What is claimed is:

1. A method for deploying an implant in a curved vessel, comprising the steps of:
providing a steerable catheter having a proximal end, a distal end, and an endoluminal implant carried in a distal region of the catheter, the catheter having a longitudinal axis at the distal end of the catheter, the endoluminal implant having a leading edge and a trailing edge;
advancing the catheter into a curved region of a vessel having a centerline;
steering the catheter to substantially align the longitudinal axis with the centerline of the vessel at the region where the implant lies within the vessel; and
deploying the implant to achieve substantially uniform wall contact with the endoluminal surface of the vessel.

2. The method of claim 1, wherein the vessel is the aorta.

3. The method of claim 2, wherein the step of advancing the catheter into a curved region of a vessel comprises advancing the catheter into the aorta arch.

4. The method of claim 3, wherein the catheter is advanced into the ascending aorta upstream of the innominate artery.

5. The method of claim 3, wherein the catheter is advanced into the aortic arch downstream of the innominate artery.

6. The method of claim 3, wherein the catheter is advanced into the aortic arch downstream of the left subclavian artery.

7. The method of claim 3, wherein the aortic arch has an aortic dissection and wherein the dissection has an entry point, wherein the leading edge of the implant substantially overlaps the entry point of the aortic dissection.

8. The method of claim 3, wherein the aortic arch has atheroma, and wherein the implant is deployed to retain the atheroma between the implant and the endoluminal surface of the aorta.

9. The method of claim 1, wherein the catheter further comprises a control member attached to a point on the catheter near the distal end and extending proximally from the point of attachment, and wherein the step of steering the catheter further comprises the step of withdrawing the control member to cause the distal end to curve.

10. The method of claim 9, wherein the control member is attached to a point on the circumference of the catheter.

11. The method of claim 1, wherein the endoluminal implant further comprises a longitudinal adjustment member attached on the implant near the leading edge.

12. The method of claim 11, wherein the longitudinal adjustment member attached on the implant near the leading edge extends to a point of attachment near the trailing edge.

13. The method of claim 11, wherein the adjustment member is releasably attached on the implant.

14. The method of claim 11, wherein the adjustment member is fixedly attached on the implant.

15. The method of claim 1, wherein the catheter further comprises an elongate sheath slideably covering the endoluminal implant, and wherein the step of deploying the implant further comprises the step of sliding the sheath proximally to release the endoluminal implant.

16. A medical device for deploying an implant in a curved vessel, comprising:
an elongate member having a proximal end and a distal end;
an endoluminal implant releasably carried near the distal end of the elongate member; and
a control member having a distal end attached at a point near the distal end of the elongate member and extending proximal from the point of attachment,
wherein the control member causes a distal region of the catheter to bend when an axial displacement is applied to the control member.

17-26. (canceled)

27. A method for deploying an implant in a curved vessel, comprising the steps of:
providing a catheter having a proximal end, a distal end, and an endoluminal implant carried in a distal region of the catheter, the catheter having a longitudinal axis at the distal end of the catheter, the endoluminal implant having a leading edge and a trailing edge and further comprising a longitudinal adjustment member attached on the implant near the leading edge and extending proximally;
advancing the catheter into a curved region of a vessel having a centerline;
deploying the implant; and
moving the adjustment member to adjust the orientation of a plane defined by the leading edge of the endoluminal implant so that the endoluminal implant achieves uniform wall contact with the endoluminal surface of the vessel where the endoluminal implant engages the lesser curvature of the vessel.

28-44. (canceled)

45. A method for deploying an implant in a curved vessel, comprising the steps of:
providing a catheter having a proximal end, a distal end, and an endoluminal implant carried in a distal region of the catheter, the catheter having a longitudinal axis at the distal end of the catheter, the endoluminal implant having a leading edge and a trailing edge;
advancing the catheter into a curved region of a vessel having a centerline;
adjusting the orientation of a plane defined by the leading edge of the endoluminal implant; and
deploying the implant so that the endoluminal implant achieves uniform wall contact with the endoluminal surface of the vessel where the endoluminal implant engages the lesser curvature of the vessel.

46-63. (canceled)

64. A medical device for deploying an implant in a curved vessel comprising:
an elongate member having a proximal end and a distal end;
an endoluminal implant releasably carried near the distal end of the elongate member, the endoluminal implant having a leading edge and a trailing edge; and
a longitudinal adjustment member comprising a distal segment attached on the implant near the leading edge and extending proximally,
wherein the adjustment member causes the leading edge of the endoluminal implant to bend into uniform wall contact with the endoluminal surface of the curved vessel.

65-74. (canceled)

75. A method for deploying an implant in a curved vessel, comprising the steps of:
providing a steerable catheter having a proximal end, a distal end, and an endoluminal implant carried in a distal region of the catheter, the catheter having a longitudinal axis at the distal end of the catheter, the endoluminal implant having a leading edge and a trailing edge;
advancing the catheter into a curved region of a vessel having a centerline;
steering the catheter to substantially align the longitudinal axis parallel with a tangent to the centerline of the vessel at the region where the implant lies within the vessel; and
deploying the implant to achieve substantially uniform wall contact with the endoluminal surface of the vessel.

76-85. (canceled)

86. A method for deploying an implant in a curved vessel, comprising the steps of:
providing a steerable catheter having a proximal end, a distal end, and an endoluminal implant carried in a distal region of the catheter, the catheter having a longitudinal axis at the distal end of the catheter, the endoluminal implant having a leading edge and a trailing edge;
advancing the catheter into a curved region of a vessel having a centerline;
steering the catheter to substantially align the longitudinal axis parallel with a tangent to the wall of the vessel at the region where the implant lies within the vessel; and
deploying the implant to achieve substantially uniform wall contact with the endoluminal surface of the vessel.

87-96. (canceled)
